(19) United States
(12) Patent Application Publication
(10) Pub. No.: US 2009/0254175 A1
(43) Pub. Date: Oct. 8, 2009

(54) Valved stent for chronic venous insufficiency

(76) Inventors: Rodolfo C. Quijano, Laguna Hills, CA (US); Hosheng Tu, Newport Beach, CA (US)

Correspondence Address: HOSHENG TU, 15 RIEZ, NEWPORT BEACH, CA, 92657-0116, US

(21) Appl. No.: 12/080,537

(22) Filed: Apr. 3, 2008

(51) Int. Cl. A61F 2/06

(52) U.S. Cl. 623/1.24

(57) ABSTRACT

The invention discloses a valved stent and process of manufacture for treating chronic venous insufficiency having the geometry of the supporting frame and its coupling to the membrane of a specific geometry that provides the valvular mechanism for optimal function. The membrane may comprise a decellularized pericardial tissue via chemical treatment with cholic acid or bile salts and crosslinked.

### FIELD OF THE INVENTION

This invention discloses a valved stent for the replacement of absent or destroyed or correction of incompetent venous valves. More specifically, the invention discloses the geometry of the supporting frame and its coupling to the membrane of specific geometry that provides the valvular mechanism for optimal function.

### BACKGROUND OF THE INVENTION

Being somewhat different in structure than the arteries, veins are specifically designed to allow blood flow in one direction only, toward the heart, and this is only possible by the presence of numerous valves found along the lumen of most veins. Only in the vena cava, the iliac veins and the portal system of veins in the liver valves absent. The valves form an essential part of the pumping system that is returning blood to the heart from the lower limbs against the force of gravity and thus protect the peripheral tissues from the retrograde pressures of the column of blood in the veins when the person is upright. Venous valves are very unique, formed usually by two, sometimes three gossamer-like cusps or leaflets which, despite their delicate appearance, are surprisingly strong. These cusps are supported by venous walls with inner lining endothelium that has non-thrombogenic properties and releases non clotting factors like plasmin. The vein walls are very thin but are capable of considerable dilatation or contraction. When the person is standing or upright, the veins are maximally distended. Their diameter has increased to several times their diameter when the patient and the limbs are horizontal.

The veins are very flexible such that if limbs are elevated, all the blood can leave the vein and they collapse into thin ribbon-like flat shape that does not allow suction or siphoning of blood along its length. Although their endothelium is non-thrombogenic, blood flow along a vein is very slow and periods of stasis may encourage and promote thrombosis more easily than arteries. There are mechanisms to minimize this occurrence such as the production of prostacyclins that discourage aggregation of platelets, and actively forming fibrinolysins such as plasmin capable of dissolution of thrombi and clots.

The numerous and powerful muscles of the lower limb require copious blood flow to perform the great number of activities than require energy. These muscles contain numerous sinuses distributed within their veins that can gather blood from the small veins and venules to be used when the next muscle contraction occurs. The greater the effort the muscle performs, the grater the venous sinuses will be. On contraction the venous sinuses will empty into the main large veins. The pumping chambers within muscles, emptied by muscle contractions are highly effective and will match the blood supply required by the working muscle. The lower limb in the human has a system of deep and superficial veins that connect to each other by conduits called perforator veins. When standing still, the veins fill to capacity in about 30 seconds. With contraction of leg muscles, the deep veins are compressed and empty upwards, the only direction allowed by venous valves. With relaxation the muscles, the veins protected from reflux by the venous valves, are slack and refill slowly by arterial flow across capillary beds and venules. Blood is then carried back to the heart by the big veins, that have less or no valves, and the thoracoabdominal pressure which can exert a brief but powerful downward thrust normally resisted by valves, a negative thoracic pressure driven by the diaphragm will draw blood toward the heart, phasic respiratory ebb and flow in venous return is present normally.

From its onset, chronic venous insufficiency even in the early stages makes itself evident in the development of telangectasia, also called hyphen web veins or “spider-veins”. They lie superficially in the dermis, are usually 1 mm or less in diameter, are impalpable and render the overlying skin purple or bright red. Progress of the disease is marked by the appearance of varicose veins or varicosities bulging through the skin in tortuous paths along and around the leg. These varicosities are often unsightly, and patients complain of aching, tiredness, restless legs, bocturnal cramps and itching, Such symptoms develop because of venous hypertension and are present in about half of the population, although physicians believe there is no relation to varicosities. A proportion of the patients with varicosities go on to develop the complications of chronic venous insufficiency, such as lipodermatosclerosis, “ankle flare”, thrombophlebitis, hemorrhage and leg ulcers. There is no evidence at present that demonstrate that early varicose vein surgery will prevent these complications from developing.

When there is a state of inadequate venous return in the upright position that may be accompanied by venous hypertension, this is usually referred to as venous insufficiency, and as the state becomes long lasting it is termed chronic venous insufficiency (CVI). The causes for this state can be varied, generally the venous pump is overwhelmed by downflow in incompetent superficial varicose veins (failed valves). Valves may have failed due to thrombosis or by their leaflets becoming stretched or shrunk becoming unable to coapt or appose to each other to provide closure that impedes retrograde flow of blood normally. Inactivity due to arthritis or paralysis can cause the malfunction of these valves.

The visible and palpable signs of CVI on the lower limb span from spider veins, a network of bluish superficial very thin veins, to tortuous varicose veins with occasional saccules along the vein path. The varicose vein may become adherent to the thin overlying superficial skin, and stretched by pressure the dark venous blood will be noticeable through the skin, and be vulnerable to hemorrhage by minor trauma. Thinning of the dermis ensues associated with poor blood supply that makes the skin very susceptible to trauma. The smallest scratch will rupture the skin that has little normal blood flow, and the rupture becomes an ulcer that is unsightly, ill-smelling, painful and difficult to heal. Venous ulcers are notoriously slow to heal; one study showed that 50% of ulcers had been open for one year or more. An ulcer may heal by various applications of unguents and salves, bandaging and repeated cleaning, thus reverting to the third stage, but it can also progress and give rise to worsening conditions that may necessitate amputation of the limb. It has been determined that there are approximately 2.6 million venous stasis ulcers that require treatment in the USA yearly. Venous hypertension thus is due to the failure of the mechanism which normally lowers venous pressure upon ambulation, namely: venous reflux due to valvular incompetence (90%), which affects both superficial and deep veins and is due to primary valvular insufficiency, as in varicose veins, or post-thrombotic damage (10%) that initially was venous obstruction. As such, most treatments to date address the symptoms not the root cause of the disease.

The venous system extends through the heart to the lungs, and although the vessels exiting the heart from the right side in the direction of the lungs are termed pulmonary trunk and arteries, they carry venous blood and their structures are more like that of veins than of arteries. The valve at the exit of the right side of the heart, the pulmonic or pulmonary valve, may be at times dysfunctional, deformed, or in congenital errors such as pulmonary atresia, be absent. This is another form of venous insufficiency. This condition can be corrected with a replacement valve of biological nature preferentially because if not corrected can be fatal.

Blood pressure or vascular pressure refers to the force exerted by circulating blood on the walls of blood vessels, and constitutes one of the principal vital signs. The pressure of the circulating blood decreases as blood moves through arteries, arterioles, capillaries, and veins; the term blood pressure generally refers to arterial pressure, i.e., the pressure in the larger arteries, arteries being the blood vessels which take blood away from the heart. Typical values for a resting, healthy adult human are approximately 120 mmHg systolic and 80 mmHg diastolic. Venous pressure is the vascular pressure in a vein, typically less than 30 mmHg, or in the atria of the heart. It is much less than arterial pressure, with common values of 5 mmHg in the right atrium and 8 mmHg in the left atrium. Therefore, the impact of blood pressure on a stented valve in an arterial vessel is substantially higher than that in a venous vessel. The construct, material and configuration of a valved stent for treating chronic venous insufficiency are quite different from those properties required for treating an arterial valve problem.

Although a great variety of treatments have been tried for centuries to correct venous insufficiency and mostly the end stages of tissue damage and ulceration, no treatment has ever provided reliable and lasting improvement of the condition. For centuries, salves and unguents, bandaging and other external applications of a host of medications were the practice, but that ameliorated symptoms only temporarily. Physicians turned to surgical procedures in an attempt to restore function of incompetent valves. Once again multiple procedures were attempted in valves that were dysfunctional because of dilated aggers, increased luminal diameter secondary to dilation of the veins that results in reflux simply because the valve cusps do not meet, or because of stretched or slightly shrunk leaflet tissue. It was noted that sometimes competence was restored merely by the venospasm during dissection. This observation prompted some to use an external band to reduce the diameter of the vein at the level of the base of the valve or the whole length of the valve and restore competence.

Thus, Dacron, polytetrafluoroethylene (PTFE), and fascial sleeves wrapped around were used to create the vein cuffs. Lane, in U.S. Pat No. 7,335,214 issued on Feb. 26, 2008, the entire contents of which are appended herein by reference, teaches of such banding device. Internal surgical procedures which necessitated dissecting the vein transversely or longitudinally to access the valvular mechanism and apply the corrective measures to ensure coaptation of the leaflets and restore competence have been developed. These procedures require extreme expertise, and few physicians are so well trained; pose a great risk of thrombosis and thromboembolism, a frequent event when a vein is dissected or reanastomosed, and an event that can prove to be fatal at times if it progresses to embolize the thrombus in the lung.

Others, translocated venous valves that are also found in the arms, up to the level of the axilla, to the lower limbs. The vein valve in the arm experiences different flows and pressures than the vein valve in the lower limb; this precipitates the reversal to incompetence of the transplant. A chemically preserved bovine jugular vein with an integral venous valve was proposed and used by Quijano, in U.S. Pat. No. 7,159,593 issued on Jan. 9, 2007, the entire contents of which are appended herein by reference, for surgical replacement of defective vein valves in patients suffering from chronic venous insufficiency. When implanted as an interposition in common femoral veins, it functioned well but thrombi developed at the line of suture. The leaflets were clean and thin, having shown good motion by ultrasound scanning.

The trauma of surgery in venous disease is well known to steeply increase the risk of thrombosis, thromboembolism and pulmonary embolism. Later the same chemically preserved biological jugular vein valve mounted in a specially designed shape memory alloy was implanted into the femoral vein by transluminal percutaneous catheter means. Of very few implants in the lower limb veins, 80% failed due to stent weakness without fracture. The frame unable to maintain its programmed diameter in the longitudinal direction, namely not having sufficient radial strength to maintain the diameter of the vein as needed along the vein course, narrowed the inflow orifice causing hemodynamic detriment that precipitated thrombosis and failure. However, the same bovine jugular venous valve mounted in a Pt—Ir stent was implanted by Bonhoeffer [JACC 2002;39:1664-1669] through the femoral vein into the right ventricular outflow tract pulmonary trunk artery to replace the pulmonary valve. This is the first cardiac valve ever implanted in a human transluminally by catheter successfully. The number of implants now approaching 3,000 with good function and not too high complication rate. Valves implanted by catheter means through arteries have proliferated in the last few years.

Cribier in U.S. Pat. No. 6,908,481 issued on Jun. 21, 2005, the entire contents of which are appended herein by reference, discloses a valve mounted in a stainless steel stent to be placed in the aortic root position through a retrograde path starting in the femoral artery below the groin and expanding to the designed diameter. Seguin in U.S. Pat. No. 7,329,278 issued on Feb. 12, 2008, the entire contents of which are appended herein by reference, uses a shape memory metal stent with a mounted biological valve, delivered transfemorally in retrograde fashion through the aorta into the aorto-ventricular junction where it is expected to self-expand and function. Both of these methods used in the arterial system where pressures and flows are distinctly higher suffer from total proper function, failing often because of peri-valvular reflux, due to incomplete seal by the stent.

Inventions of devices and methods for least invasive treatment of venous insufficiency to eliminate need of open surgical treatment are not as plentiful as those used for correction of arterial problems. Those devices span from delivery catheters that will deploy appliances or prosthesis to sites in a vein where a defective or non closing valve may be. The devices may by design grasp the poorly functioning leaflets and force them to appose and provide competence to the valve by curtailing reflux of blood through the valve to delivery systems for stents with some moving parts that would attempt to reproduce the function of a venous valve. Laufer et al in U.S. Pat. No. 6,149,660 issued on Nov. 21, 2000, the entire contents of which are appended herein by reference, shows a device that through a delivery catheter approaches the free edges of the venous valve in the leg and applies a clip appliance that is affixed to the valve rendering it competent.

Peripheral stents delivered intraluminally to various vessels in the body have been used for over a decade. In the last decade a few have included valves in some of these stents for use in the venous system to correct venous valve insufficiency. Pavcnick in U.S. Pat No. 6,200,336 issued on Mar. 13, 2001, the entire contents of which are appended herein by reference, discloses a multiple sided device, the preferred embodiment being square or rhomboid, made from wire that in a folded configuration becomes a self expanding stent at times comprising barbs to anchor it to the vessel luminal wall to prevent its migration and possessing a flat membrane of biocompatible material that attempts to function as a valve. Both the stent and the material fail to provide the function of a venous valve, the stent configuration does not provide stability within a vessel to maintain it parallel to the flow of blood, as it tilts and disturbs the normal flow of venous flow, and allows reflux, allows peripheral or peri-stent valvular leakage or reflux. The membrane that is not fixed or cross-linked and is configured in an isosceles triangular fashion rather that the geometry of a venous valve, does not provide for the proper fluid vector fields to minimize thrombosis and to be able to withstand the stresses and strains found in the limb venous system.

The issues encountered with the devices disclosed to the present time, suggest that there is a need for a valved stent of the possible smallest profile, that can be delivered with minimal trauma, to venous vessels of an extensive size range, where it can expand to fit the vessel diameter and resists any changes, without deformation, in the presence of vessel centripetal forces of contraction, and of sufficient dimensions at any vessel size to prevent tilting or tumbling over, in addition to provide enough force radially from its longitudinal axis, to maintain it in the original deposited site, that is to prevent its migration from the site.

### SUMMARY OF THE INVENTION

The invention herein disclosed would overcome major issues and complications that the heretofore proposed devices for the correction of chronic venous insufficiency. It is one object of the invention to provide a valved stent having a specially designed lattice structure, fabricated from one piece tube of temperature sensitive shape memory alloy, incorporating within a very specifically designed geometrical membrane of biocompatible material of specific thickness that will approximate quite closely the configuration of human and animal venous valves. It is very important that the coupling of the specially aggregated lattice members and the membrane shape that is obtained by consideration of the course of some of the members that form the lattice of the stent when fully expanded be accomplished in a very orderly manner for each size or diameter well as length of valve assembled as well as the configuration of valve assembled. Thus, in this manner the shape of the venous valve is defined and experiments suggest that the valve meets the desired specifications in flow control, allowing quasi-laminar flow to pass through the valve, minimizing turbulence that is deleterious and leads to thrombus formation, and providing ample coaptation to ensure the competency of the valve under conditions of diameter changes in the agger or better described as dilatation of the “annulus” of the venous valve.

It is another object of the present invention to provide the configurations permutations of the device in terms of the holding frame of the valvular mechanism as well as the configuration of the tissue valvular mechanism itself as both bear high importance in the performance of the valved stent as a venous valve.

The device has a first configuration of the holding frame wherein in the expanded final form after implantation in a venous lumen, the reticular frame is in the form of a cylinder of a specified length depending on the specified diameter required for a defined one by the diameter of a limb vein in need of a valve to maintain blood flow continuation in the direction of the heart. This cylinder with a specific wall thickness such that there is a hollow central passage through which the venous blood will flow, and the thickness of the stent is the thickness of the members that form the lattice of the cylinder wall. The cylinder then has a circular inflow aspect through which blood returning from the feet would enter, and a circular outflow aspect that allows blood flow continuity in the direction toward the heart only for in between these two aspects a valvular mechanism specifically configured to fit certain members of the lattice and to open as blood flows through it, but closes as the flow begins to reverse through the cycle provided by the rhythmic thoraco-abdominal exertion of pressure on the vein and the withdrawal of that pressure by the change of diaphragm position during respiration that reverses the pressure and stimulates flow in the direction of the heart.

The valvular mechanism provided consists of one piece of membrane of a very specific shape defined by the stent lattice members configuration for a specific size of vein valved stent fabricated, this one piece membrane's geometric configuration is arrived by a well defined algorithm that will be the essence of this invention. This algorithm would vary with the size of valved stents to be produced as well as whether the configuration of the valvular mechanism is formed by three equal leaflets or the valve is bileaflet in configuration. Venous valves found in mammals, quadrupeds, or bipeds, and in the human venous system can be found to have both of these configurations in general.

In some aspects, there is provided a biological tissue material or tissue sheet for membrane or leaflet construct. In a preferred embodiment, the natural tissue or tissue sheet material is selected from a group consisting of porcine pericardium, bovine pericardium, equine pericardium, ovine pericardium, caprine pericardium, fascia lata, dura mater and the like.

In another embodiment, the tissue sheet material is crosslinked with a crosslinking agent or with ultraviolet irradiation, wherein the crosslinking agent may be selected from the group consisting of genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, and combinations thereof.

Some aspects of the invention provide a process for the production of a decellularized pericardial membrane, sheet or strip (collectively coded as pericardial tissue), to fashion the valvular mechanism comprising: a pericardium tissue sheet having cells and extracellular matrix; subjecting the sheet to a solution containing bile acid or bile salts which effect the solubilization of cell membranes of the cells present in the tissue sheet; removing the solubilized cell membranes by flushing the tissue sheet with filtered water; and treating the tissue sheet with a crosslinking agent. In one embodiment, it is provided a decellularized pericardial tissue produced by the process of the present invention.

The decellularized pericardial tissue would contain less cellular residues because the solubilized membrane detaches from the surface of the extracellular matrix inside the tissue sheet and is relatively easy to remove for example, by flushing with filtered water. Removal of the cells would provide an additional masking of the material to the body's immunological protection, or foreign tissue rejection, a beneficial effect to be added to the masking already provided by crosslinking with a fixative such as glutaraldehyde. This treatment may also result in a lowering of the thrombogenic potential of the membrane, a define need when these membranes will be in contact with venous blood, as is known that foreign materials in the venous system tend to thrombose because of their nature and because of the low flows and stagnation normally occurring in that system.

In one embodiment, the tissue sheet is selected from a group consisting of porcine pericardium, bovine pericardium, equine pericardium, ovine pericardium, fascia lata, and dura mater. In another embodiment, the crosslinking agent is selected from a group consisting of genipin, epoxy compounds, dialdehyde starch, glutaraldehyde, formaldehyde, dimethyl suberimidate, carbodiimides, succinimidyls, diisocyanates, acyl azide, and combinations thereof.

Some aspects of the invention provide a process for sterilization with increased fixative concentration and low molecular weight solvents. As the device must be rinsed with copious volumes of physiological saline, it is found that not all fixative, very often glutaraldehyde fixative, can be removed before implantation. Small amount of the fixative will remain in the tissue. In those areas (the stagnant areas) where there is contact between the chemically preserved tissue and the wall of the vessel, a slight irritation occurs but there is also a combination in situ of fibrin from blood. Fibrin is known to react with glutaraldehyde that is known to produce a mucilaginous viscous material that is also known to be an adhesive to bind different tissues together. Thus, in the areas where the glutaraldehyde fixed tissue is attached to the frame, becomes the margin of attachment of the venous valve to the vessel wall and becomes glued or adhered to such wall with fibrin-glue that will with time increase in thickness forming a peri-valvular seal, thus preventing reflux from the periphery of the valve. This gluing effect attaches the valved sent firmly to the vessel wall and opposes due resistance to migration of the device in either direction. This glue-glutaraldehyde complex differentiates this device from all others mentioned where the tissue is relatively inert and cannot form the mucilaginous byproduct to ensure the attachment.

An algorithm was found to describe the shape of each leaflet and combined with one or two others to result in the entire valvular mechanism. The shape of the leaflet is dependent on the shape of the members of the lattice (struts) forming the stent, which in turn is dependent on the radius of the valved stent desired based in turn on radius of the failed vein valve at the agger of the valve, equivalent to the radius or diameter of the vein containing that valve.

This algorithm is similar to those that can define azimuth and declination in the celestial sphere in astronomy. The line joining the tips of two zigs on the same horizontal plane of the lattice of the cylindrical stent of a certain radius R, is the chord S of the circular arc of the cylinder. Their relationship can better be described by the following equation

T=(S/R)×(180/π)
where T is the length of chord at any specific axial set-point upstream of the free margin, a first end of the chord being the first mounting point on a right-spiral strut and a second end of the chord being the second mounting point on an adjacent left-spiral strut, both mounting points being at the same axial level of that specific set-point.
R is a radius of the valved stent, and
S is the length of chord of the circular arc of the stent cylinder at any specific set-point axially spaced upstream from the free margin.

Some aspects of the invention provide a valved stent comprising: a cylindrical meshed stent component with lattices, the stent component having an inflow section and an outflow section with respect to flow direction, each of the inflow and outflow sections having right-spiral struts, left-spiral struts and a strut joint at a crossing of any of the right-spiral struts and any of the left-spiral struts; and a leaflet component with at least two identical leaflets, each leaflet having a free margin and two margins of attachment, wherein a first margin of attachment is mounted on a right-spiral strut and a second margin of attachment is mounted on an adjacent left-spiral strut, the free margins of the at least two leaflets coapting to form a one-way flow valve. The valved stent of the present invention is configured for treating chronic venous insufficiency in a venous circulation system that has relative low blood pressure, for example, a blood pressure of less than 30 mmHg.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 shows a valved stent in absence of a mounted valve for illustration purposes, having an inflow section and an outflow section.

FIG. 2 shows a cross-sectional view of the section I-I of FIG. 1, showing a strut covered with a tissue strip.

FIG. 3 shows a valved stent with a meshed stent component and a leaflet component, wherein the leaflet component is coupled onto the stent component and becomes an integral part of the valved stent.

FIG. 4 shows a cross-sectional view of the section II-II of FIG. 3, showing a portion of leaflet wrapped over a tissue-strip covered strut.

FIG. 5 shows a leaflet membrane component of the valved stent of the present invention, showing three identical leaflets and their extended flat material from the leaflets in the leaflet component.

FIG. 6 shows a cylindrical meshed stent component of the valved stent, including an inflow section and an outflow section.

FIG. 7 shows a tubular meshed stent component of the valved stent, having a cylindrical inflow section and a bulged outflow section where a valvular mechanism is located.

FIG. 8 shows means for trimming or cutting the leaflet membrane for mounting on the meshed stent component of the present invention; (A) showing a cut leaflet in a 3-D configuration, and (B) showing a cut leaflet in a 2-D top-view flat configuration.

### DETAILED DESCRIPTION OF EXEMPLARY EMBODIMENTS

The following detailed description is of the best presently contemplated modes of carrying out the invention. This description is not to be taken in a limiting sense, but is made merely for the purpose of illustrating general principles of embodiments of the invention.

Phifer and associates (Am J Surg 1989;157:588-592) evaluated 22 valvular xenografts placed in the inferior vena cavas of 22 dogs. Patency extended to 28 months in 12 valves. Patent valves functioned with minimal pressure gradients at moderate flow with no evidence of pulmonary emboli. However, the study raises questions concerning design requirements for prosthetic venous valves.

Gerlock and associates (Invest Radiol 1985;20:42-44) evaluated bioprosthetic valves surgically placed in the inferior vena cava of four canines for the treatment of nonobstructive chronic venous stasis. Valve patency and thrombus formation involving either the valve or the IVC was evaluated by serial follow-up inferior vena cavagrams in each canine. No thrombus occurred in any of the canines as observed at six months in two canines and eight months in another two canines. It appeared feasible to develop a valvular prosthesis which may be placed into the venous system for the management of nonobstructive chronic venous stasis.

Later, Gomez-Jorge and associates (J Vasc Interv Radiol 2000;11:931-936) evaluated a system for potential use in the treatment of chronic venous insufficiency by using percutaneous techniques. A segment of a glutaraldehyde-fixed bovine external jugular vein with valves was trimmed and sutured to a nitinol stent. Eleven bioprostheses were deployed in 11 animals (in the IVC or right external iliac vein). Deployments of the bioprostheses were successful in nine of 11 swine. Complications included hemarthrosis, death, and bioprosthesis thrombosis immediately after deployment. They concluded that development of a venous bioprosthesis that can be placed percutaneously may have important clinical applications as an endovascular treatment for chronic venous insufficiency when it is due to valvular incompetence.

Taheri and associates (Am J Surg 1988;156:111-114) reported that medical treatment of venous insufficiency syndrome has been associated with a high incidence of failure. They evaluated a prosthetic vein valve in ten dogs without anticoagulants. It was concluded that between 3 and 8 months after insertion, ascending and descending venography revealed patency and competency of these valves.

Boudjemline and associates (Med Sci Monit 2004;10:BR61-66) evaluated the feasibility and safety of percutaneous implantation of a balloon-expandable valved stent in the inferior vena cava. A valve harvested from a bovine jugular vein, preserved in glutaraldehyde and mounted in a stent, was evaluated in six lambs. All valved stents were successfully implanted in the desired position. No early or late migration of the stent was noted in any animal. All valves were perfectly competent at the time of implantation. At 2 months, none of the valves were functional. The inferior vena cava was occluded at the site of valve insertion and collateral circulation was present in all animals. At autopsy, the valved stent was completely occluded. It was concluded that valve implantation is feasible in the venous system through a percutaneous approach. The function of this valve in that position is limited by the absence of a high pressure gradient.

Taheri and associates (Int Angiol 1989;8:7-9) investigated a sutureless prosthetic vein valve by inserting into the femoral vein or vena cava of mongrel dogs. The sutureless prosthetic vein valve externally supported by platinum or titanium appeared a promising development in the treatment of venous insufficiency.

A “tissue material” refers to a biomedical material of biological tissue origin which might be decellularized and crosslinked to form a medical device. A tissue sheet, such as a pericardial sheet, is in a sub-group of tissue material (including sheet form and non-sheet form).

A “decellularization process” is meant to indicate the process for detaching and removing a substantial portion or all of cellular substance from cellular tissue and/or tissue matrix that contains connective tissue protein/collagen, for example, a pericardial sheet. To be used in a medical device, the cellular substance of the cellular tissue does not provide any beneficial function, but might be a source of foreign reaction.

It is one object of the present invention to provide a decellularized, crosslinked tissue sheet as raw material for manufacturing the leaflet membrane of the valved stent of the present invention. Further, the decellularization process may be accomplished via cholic acid treatment as described in details below.

Properties of Cholic Acid

Cholic acid, shown below, has an empirical formula of C₂₄H₄₀O₅.

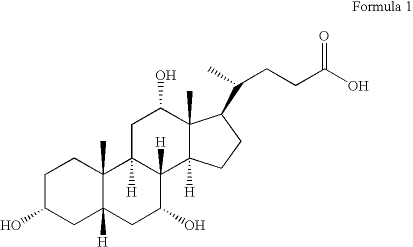

Cholic acid is, a white crystalline substance insoluble in water, with a melting point of 200-201° C. Salts of cholic acid (also broadly herein including derivatives of cholic acid) are called cholates or bile salts. Cholic acid is one of the four main acids produced by the liver where it is synthesized from cholesterol. It has active side groups (COOH and OH) and is soluble in alcohol and acetic acid. Cholic acid possesses a particular hydrogen (the singular ‘H’ shown at the left lower corner of the structure formula above). As a result, the first six-carbon ring on its right-hand side and the second six-carbon ring on its left-hand side are no longer coplanar but have a cis-configuration (a three-dimension structure). This cis-configuration of two contiguous six-carbon rings improves the detergent properties of the bile acids so they are better able to solubilize lipids.

Glycocholate is an example of a bile salt. The cholic acid forms a conjugate with taurine, yielding taurocholic acid. Cholic acid and chenodeoxycholic acid are the most important human bile acids. Some other mammals synthesize predominantly deoxycholic acid. The main use of cholic acid is as an intermediate for the production of ursodeoxycholic acid. Ursodeoxycholic acid is a pharmaceutical product which is used for several indications including the dissolution of gallstones and the treatment and prevention of liver disease. Cholic acid (broadly herein defined to include its derivatives) has many different uses in traditional Chinese medicine. Its main use is as an ingredient in the manufacture of artificial calculus bovis (artificial gallstones).

Deoxycholic acid with an empirical formula of C₂₄H₄₀O₄, is sparingly soluble in water, but soluble in alcohol and to a lesser extent acetone and glacial acetic acid. Historically deoxycholic acid was used as an intermediate for the production of corticosteroids, which have anti-inflammatory indications.

An emerging use of deoxycholic acid is as a biological detergent to lyse cells and solubilize cellular and membrane components. Some aspects of the invention relate to a process of decellularization of tissue or tissue biomaterial via delipidation as a medical device. It is suggested that cell extraction as a result of cholic acid decellularization removes lipid membranes and membrane-associated antigens as well as soluble proteins. In one embodiment, the process of delipidation or decellularization via delipidation of tissue or tissue biomaterial utilizes cholic acid, deoxycholic acid, or bile salts (including salts of cholic acid and its derivatives, such as glycocholate and deoxycholate) sufficient to delipid and subsequently decellularize the tissue biomaterial.

In a preferred embodiment, the delipidated and/or decellularized tissue or tissue biomaterial is further crosslinked (for example, through ultraviolet irradiation) or treated with a chemical agent, such as genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, and combinations thereof. Other crosslinking means may also apply to crosslink the decellularized tissue (pericardial and non-pericardial tissues) of the present invention.

Cholic acid and deoxycholic acid has a low acute toxicity, with LD₅₀ i.v. 50 mg/kg and 15 mg/kg in rabbit, respectively. In general, bile acids and salts have only a minor toxic potential when given by mouth. In large doses, they are likely to have the same effects as saponins; the main action is likely to be irritation of mucous membranes. Parenterally they are much more toxic and may cause hemolysis, a digitalis-like action on the heart and effects on the central nervous system.

Bile is a bitter, yellow to greenish fluid composed of glycine or taurine conjugated bile salts, cholesterol, phospholipid, bilirubin diglucuronide, and electrolytes. It is secreted by the liver and delivered to the duodenum to aid the process of digestion and fat absorption by emulsification of fat products in the upper small intestine. They play role of dissolving cholesterol and accretes into lumps in the gall bladder, forming gallstones. Bile's bicarbonate constituent serves for alkalinizing the intestinal contents. Bile is responsible for as the route of excretion for hemoglobin breakdown products (bilirubin). Excretion of bile salts by liver cells and secretion of bicarbonate rich fluid by ductular cells in response to secretion are the major factors which normally determine the volume of secretion. Bile acids are liver-generated steroid carboxylic acids. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis(3D Gluconoamidopropyl)deoxycholamide. Salts of bile acids are normally called bile salts.

The primary bile acids (for example, cholic and chenodeoxycholic acid) are conjugated with either glycine or taurine in the form of taurocholic acid and glycocholic acid. The secondary bile acids (deoxycholic, lithocholic, and ursodeoxycholic acid) are formed from the primary bile acids by the action of intestinal bacteria. They are soluble in alcohol and acetic acid. The lithocolyl conjugates are relatively insoluble; excreted mostly in the form of sulfate esters like sulfolithocholylglycine. Most of the bile acids are reabsorbed and returned to the liver via enterohepatic circulation, where, after free acids are reconjugated, they are again excreted.

Sung et al. in U.S. Pat. No. 6,998,418, entire contents of which are incorporated herein by reference, discloses a biological tissue configured and adapted for tissue regeneration, the tissue being characterized by reduced antigenicity reduced immunogenicity and reduced enzymatic degradation upon placement inside a patient's bode with porosity being increased by at least 5%, further comprising an angiogenesis agent, stem cells or autologous cells. Further, the biological tissue may be a bovine pericardium, an equine pericardium, or a porcine pericardium with increasing porosity of the tissue that is provided by an enzyme treatment process, by an acid treatment process, or by a base treatment process. However, the U.S. Pat. No. 6,998,418 patent does not teach the process of delipidation and/or decellularization of tissue biomaterial by utilizing cholic acid (bile acid) or bile salts.

Noishiki et al. in U.S. Pat. No. 4,806,595 discloses a tissue treatment method by a crosslinking agent, polyepoxy compounds. Collagens used in that patent include an insoluble collagen, a soluble collagen, an atelocollagen prepared by removing telopeptides on the collagen molecule terminus using protease other than collagenase, a chemically modified collagen obtained by succinylation or esterification of above-described collagens, a collagen derivative such as gelatin, a polypeptide obtained by hydrolysis of collagen, and a natural collagen present in natural tissue (ureter, blood vessel, pericardium, heart valve, etc.) The Noishiki et al. patent is incorporated herein by reference. “Collagen matrix” in the present invention is collectively used referring to the above-mentioned collagens, collagen species, collagen in natural tissue, and collagen in a biological implant preform.

In one embodiment, the crosslinker or crosslinking agent of the invention may be selected from a group consisting of genipin, its analog, derivatives, and combination thereof, epoxy compounds, dialdehyde starch, glutaraldehyde, formaldehyde, dimethyl suberimidate, carbodiimides, succinimidyls, diisocyanates, acyl azide, tris(hydroxymethyl)phosphine, ascorbate-copper, glucose-lysine, and combinations thereof.

### EXAMPLE NO. 1

Tissue Sheet Preparation via Decellularization

In one embodiment of the present invention, porcine pericardia procured from a slaughterhouse are used as raw materials. In the laboratory, the pericardia are first gently rinsed with fresh saline to remove excess blood on tissue. The cleaned pericardium before delipidation process is herein coded specimen-A. The procedure used to delipid the porcine pericardia is described below: A portion of the trimmed pericardia is immersed in a hypotonic tris buffer (pH 8.0) containing a protease inhibitor (phenylmethyl-sulfonyl fluoride, 0.35 mg/L) for 24 hours at 4° C. under constant stirring. Subsequently, they are immersed in a 1% solution of Triton X-100 (octylphenoxypolyethoxyethanol; Sigma Chemical, St. Louis, Mo., USA) in tris-buffered salt solution with protease inhibition for 24 hours at 4° C. under constant stirring. Samples then are thoroughly rinsed in Hanks' physiological solution and treated with a diluted cholic acid about 5% at 37° C. for 1 hour. In one embodiment, the cholic acid solution could be from about 2% to about 99%, preferably about 5% to about 50%. This is followed by a further 24-hour extraction with Triton X-100 in tris buffer. Finally, all samples are washed for 48 hours in Hanks' solution and the decellularized sample is coded specimen-B. Light microscopic examination of histological sections from extracted tissue revealed an intact connective tissue matrix with no evidence of cells or cellular residues.

A portion of the decellularized tissue of porcine pericardia (specimen-B) is thereafter lyophilized at about −50° C. for 24 hours, followed by soaking in glycerol-containing fluid (e.g., 75% glycerol and 25% ethanol) to obtain the decellularized dehydrated pericardia. In other experiments, the glycerol content of the glycerol-alcohol mixture may range from about 50 to 100%. In another example, a portion of specimen-B is rinsed and soaked in glycerol-containing fluid (e.g., 80% glycerol and 20% ethanol) to yield decellularized “dry” dehydrated pericardia; optionally, the decellularized dehydrated pericardium is lyophilized at about −50° C. for 24 hours to get a substantially “moisture-free” dehydrated decellularized pericardium. The dehydrated decellularized tissue or pericardial tissue can be re-constituted for medical applications. In a preferred embodiment, the decellularized tissue before lyophilization is thoroughly flushed to remove crosslinking agent, In another preferred embodiment, the decellularized tissue before lyophilization is treated with a counter-agent for a particular crosslinking agent; for example, an amine-containing compound is used to react with the excess free crosslinking agent of epoxy compounds and therefore, deactivate the excess crosslinking agent remained in the tissue.

As disclosed in U.S. Pat. No. 6,998,418, the mechanism of increasing the tissue porosity treated by a mild acidic or base (i.e., a solution pH value greater than 7.0) solution lies in the effect of [H⁺] or [OH⁻] values on the collagen fibers matrix of the decellularized tissue. Similarly, a portion of the decellularized porcine pericardia tissue is further treated with enzymatic collagenase as follows. Add 0.01 gram of collagenase to a beaker of 40 ml TES buffer and incubate the pericardia tissue at 37° C. for 3 hours. The sample is further treated with 10 mM EDTA solution, followed by thorough rinse. In one embodiment, the tissue is stored in phosphate buffered saline (PBS, 0.01M, pH 7.4, Sigma Chemical). In another embodiment, the tissue is lyophilized at about −50° C. for 24 hours, followed by soaking in glycerol to obtain the decellularized dehydrated pericardia. The decellularized dehydrated pericardial patch could be sterilized (for example, EtO sterilization) before use.

### EXAMPLE NO. 2

Tissue Sheet Preparation via Crosslinking

The decellularized tissue (specimen-B) of porcine pericardia are fixed with various crosslinking agent. The first specimen is fixed in 0.625% aqueous glutaraldehyde (Merck KGaA, Darmstadt, Germany) as reference. The second specimen is fixed in genipin (Challenge Bioproducts, Taiwan) solution at 37° C. for 3 days. The third specimen is fixed in 4% epoxy solution (ethylene glycol diglycidyl ether) at 37° C. for 3 days. The aqueous glutaraldehyde, and genipin used are buffered with PBS (0.01M, pH 7.4). The aqueous epoxy solution was buffered with sodium carbonate/sodium bicarbonate (0.21M/0.02M, pH 10.5). The amount of solution used in each fixation was approximately 200 mL for a 10 cm×10 cm porcine pericardium. Subsequently, the fixed decellularized specimens are sterilized in a graded series of ethanol solutions with a gradual increase in concentration from 20 to 75% over a period of 4 hours. Finally, the specimens are thoroughly rinsed in sterilized PBS for approximately 1 day, with solution change several times, and prepared for tissue characterization with respect to degree of crosslinking and appearance. All specimens show crosslinking characteristics per analysis of amino acid residue reactions, increased denaturation temperatures, and resistance against collagenase degradation. The epoxy compounds crosslinked specimen shows whitish translucent appearance with soft flexible feeling; the glutaraldehyde crosslinked specimen shows yellowish appearance with semi-rigid feeling; and the genipin crosslinked specimen shows dark bluish appearance with flexible feeling.

In the present invention, the terms “crosslinking”, “fixation”, “chemical modification”, and/or “chemical treatment” for tissue or biological solution are used interchangeably.

Though certain methods for removing cells from cellular tissue and/or acid treatment, base treatment, enzyme treatment to enlarge pores are well known to those who are skilled in the art, it is one object of the present invention to provide a decellularized biological scaffold chemically treated with cholic acid or salts of cholic acid (for example, bile salts) as means of decellularization having increase of porosity for future potential application in tissue regeneration. Some aspects of the invention provide a process for the production of a decellularized pericardial tissue (patch, sheet strip, and other appropriate shapes or configurations) comprising: (a) providing a pericardium tissue sheet having cells and extracellular matrix; (b) subjecting the sheet to a solution containing bile acid or bile salts which effect the solubilization of cell membranes of the cells present in the tissue sheet; (c) removing the solubilized cell membranes by flushing the tissue sheet with filtered water or other solution; and (d) treating the tissue sheet with a crosslinking agent. In one embodiment, there is provided a process for the production of a decellularized tissue graft by subjecting tissue material (in a non-sheet form) to a solution containing bile acid or bile salts which effect the solubilization of cell membranes of the cells present in the tissue material and optionally treating the tissue material with a crosslinking agent. The bile acid may be cholic acid or its derivatives whereas the bile salts may be glycocholate, deoxycholate, or other cholates.

It is another embodiment of the present invention to provide a tendon or ligament graft for use as connective tissue substitute, the graft being formed from a segment of connective tissue protein or collagen, wherein the segment is decellularized via cholic acid or bile salts and optionally crosslinked. The connective tissue protein may be collagen or pericardia tissue that is substantially devoid of cells adapted for promoting autogenous ingrowth into the graft. The process for using a tissue sheet to make a tendon or ligament graft has been disclosed by Badylak et al. in U.S. Pat. No. 5,573,784, U.S. Pat. No. 5,445,833, U.S. Pat. No. 5,372,821, and U.S. Pat. No. 5,281,422, the entire contents of which are incorporated herein by reference, which disclose a method for promoting the healing and/or regrowth of diseased or damaged tissue structures by surgically repairing such structures with a tissue graft construct prepared from a segment of intestinal submucosal tissue.

Some aspects of the invention relate to a method of repairing a tissue or organ defect in a patient, comprising (a) providing a decellularized tissue sheet material having mechanical strengths; (b) repairing the defect by appropriately placing the tissue material at the defect; and (c) allowing tissue regeneration into the tissue material. By ways of illustration, the tissue sheet material according to the disclosed process of the present invention may be placed at the defect site by suturing, stapling, connecting, or welding to the defect. Other means for placing the tissue sheet material to repair the defect is within the scope of the present invention. In one embodiment, the defect is an abdominal wall defect, a vascular wall defect, a valvular leaflet defect, or a heart tissue defect. In another embodiment, the tissue sheet material further comprises at least one growth factor selected from a group consisting of vascular endothelial growth factor, transforming growth factor-beta, insulin-like growth factor, platelet-derived growth factor, fibroblast growth factor, and combination thereof. In still another embodiment, the tissue sheet material further comprises ginsenoside Rg₁, ginsenoside Re, at least one bioactive agent.

The decellularized pericardial tissue of the present invention is particularly useful as a medical device in orthopedic applications. In one embodiment, the device is used for repair of rotator cuff or strained ligaments and tendons. In another embodiment, the device is used as slings for patients with detrusor dyssynergy that causes urinary stress incontinence. The patients are prone to urinate or void every time they sneeze or dance or do some stressful activity because the slings caused by pelvic floor muscle (detrusor weakness) cannot hold the urethra at a proper angle and patient would void against his/her will. In a further embodiment, the device could be used as a membrane for burns or to cover and help the healing of venous or arterial ulcers or diabetes ulcers.

The decellularized pericardial tissue of the present invention is also useful as a medical device to repair chemical burns in the conjunctiva of the eye, to repair vessels large or small, to repair vesicles such as the bladder when torn, or as general surgical reconstruction material. In one embodiment, the pericardial tissue may be used to fabricate or repair tympanic membranes, as a fascia lata substitute and possibly other uses. Fascia lata or dura mater could be prepared in the same manner or following the same process of the present invention. The pericardial tissue may be in a form of sheet, patch or strip. The pericardial tissue may also be in a shape of square, circle, rectangular or other configurations.

Valved Stent Construct

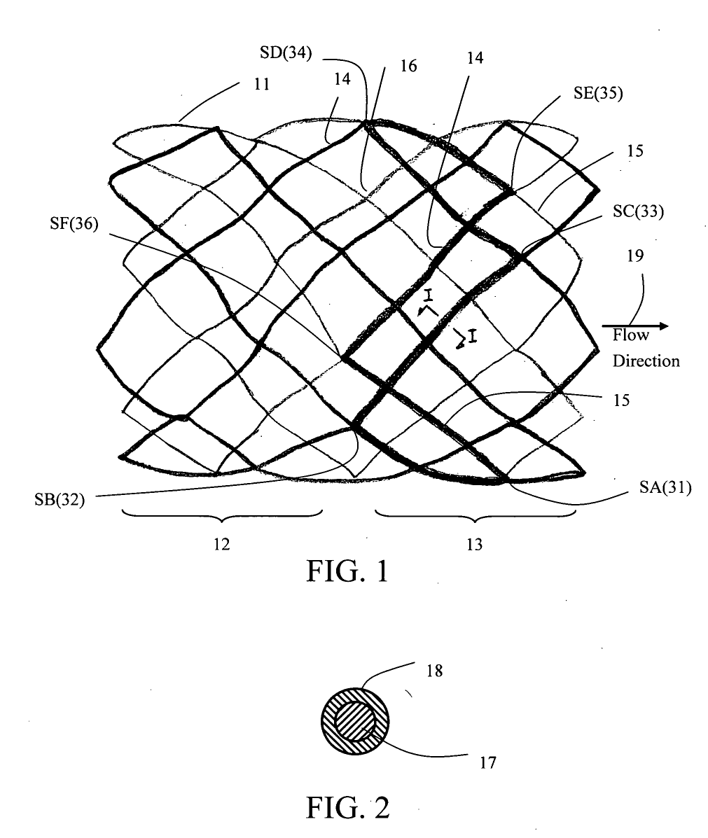

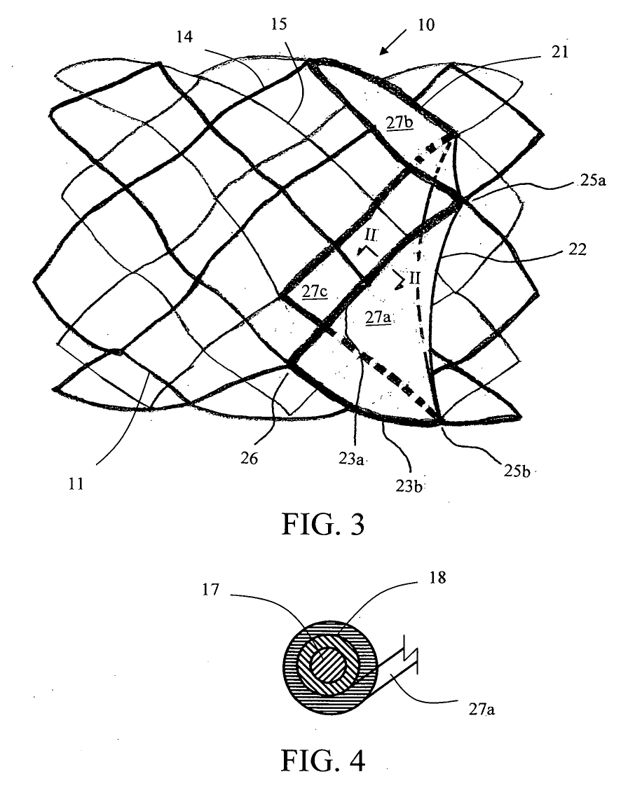

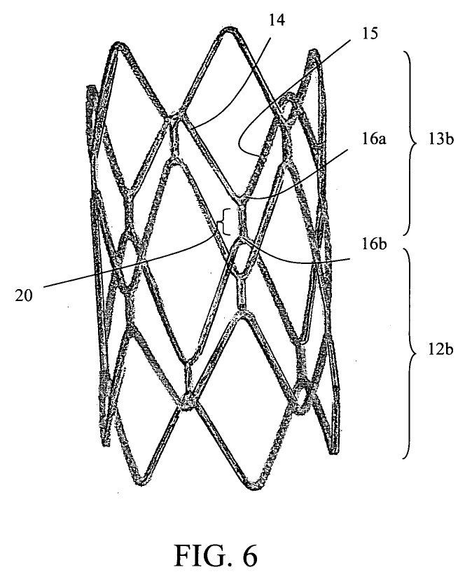

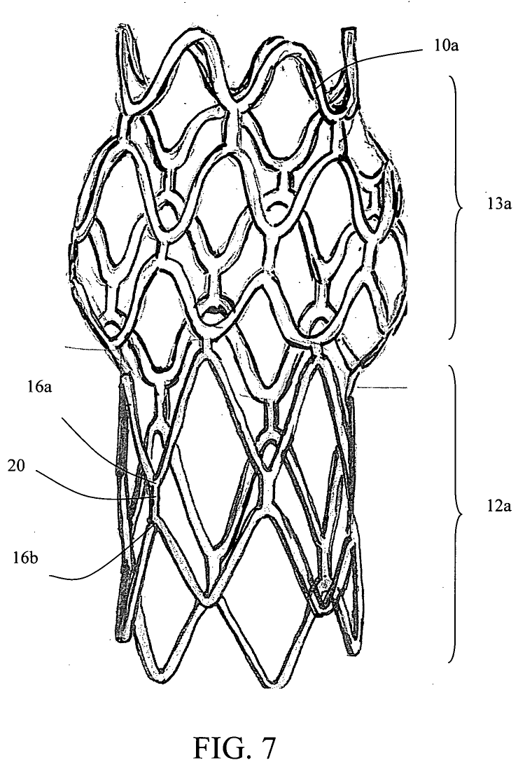

FIG. 1 shows a valved stent without the leaflet component whereas FIG. 3 shows a valved stent with a leaflet component for illustration purposes. Some aspects of the invention relate to a valved stent (10) comprising at least two components. The valved stent comprises a cylindrical meshed stent component (11) with lattices, the stent component having an inflow section (12) and an outflow section (13) with respect to flow direction (19), each of the inflow and outflow sections having right-spiral struts (14), left-spiral struts (15) and a strut joint (16) at a crossing of any of the right-spiral struts (14) and any of the left-spiral struts (15). In some embodiments, the strut joints (16a, 16b) may be linked with a vertical (axial) strut segment (20) as shown in FIG. 6 or FIG. 7.

The portion of the strut (lattice) or splines that would have received the leaflet is preferably covered with a flat thin strip (such as a pericardium strip). FIG. 2 shows a cross-sectional view of the section I-I of FIG. 1, showing a strut (17) covered with a tissue strip (18). The said portion of strip-covered strut is shown with thick lines in FIG. 1 and may include any splines section between points SA (31) and SB (32), points SB (32) and SC (33), points SC (33) and SD (34), points SD (34) and SE (35), points SE (35) and SF (36), and points SF (36) and SA (31).

FIG. 3 shows a valved stent (10) with a meshed stent component (11) and a leaflet component (21) having three leaflets (27a, 27b, 27c), wherein the leaflet component is coupled onto the stent component at those splines section from points SA (31) to SB (32), from points SB (32) to SC (33), from points SC (33) to SD (34), from points SD (34) to SE (35), from points SE (35) to SF (36), and from points SF (36) to SA (31). The leaflet component after mounting becomes an integral part of the valved stent. The leaflet component is usually mounted at about the outflow section. The inflow section is sized and configured of sufficient dimensions at any vessel size to prevent tilting or tumbling over, in addition to provide enough force radially from its longitudinal axis, to maintain it in the original deposited site, that is to prevent its migration from the site. In one embodiment, the axial length of the inflow section is about twice or more of the length of the outflow section.

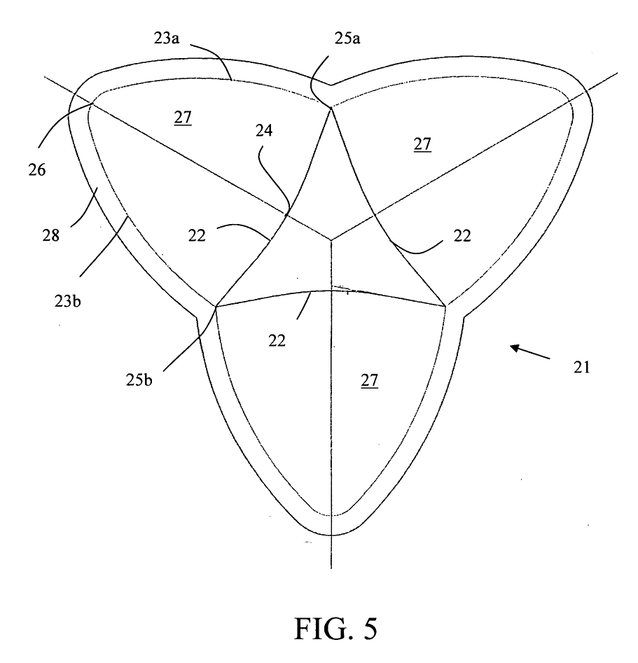

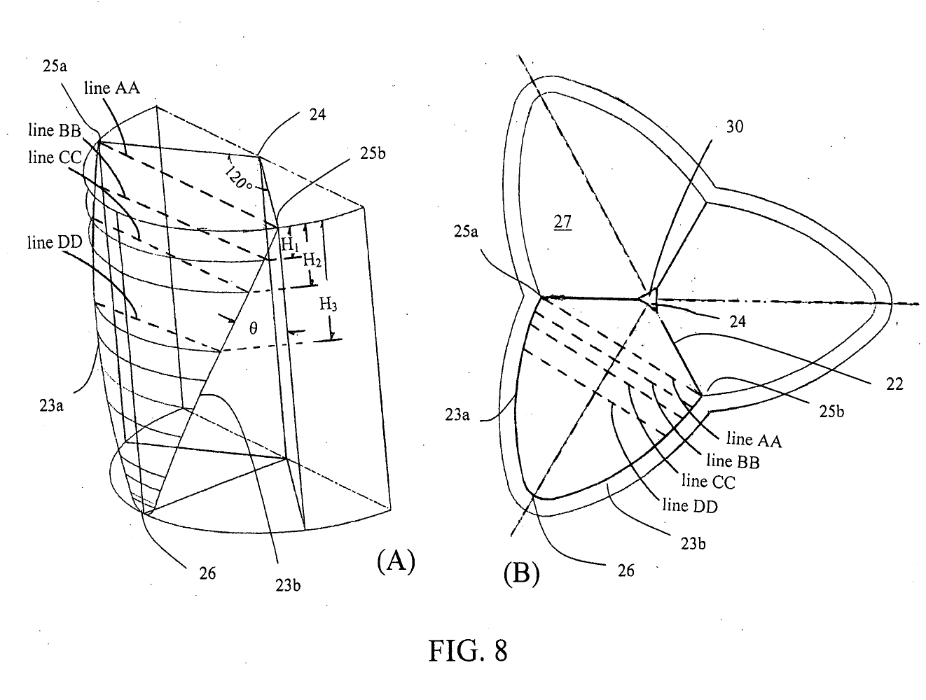

FIG. 4 shows a cross-sectional view of the section II-II of FIG. 3, showing a portion of leaflet (27a) wrapped securely over a tissue-strip (18) covered strut (17). The portion of leaflet that wrapped over the covered strut segment is the extended flat material (28) alone. The usable effective area of the leaflet of the present invention does not include the extended flat material (28) shown in FIG. 5 or FIG. 8B.

The valved stent further comprises a valvular membrane or leaflet component (21) as shown in FIG. 5. The leaflet component has at least two identical leaflets (27), each leaflet having a free margin (22) and two margins of attachment, wherein a first margin of attachment (23a) is mounted on a right-spiral strut of the meshed stent component and a second margin of attachment (23b) is mounted on an adjacent left-spiral strut, wherein a first end of the first margin of attachment meets a second end of the second margin of attachment at the strut joint of the above-mentioned right-spiral strut and the left-spiral strut, the free margins of the at least two leaflets coapting to form a one-way flow valve.

In one embodiment, the leaflet of the valved stent of the present invention comprises a middle free-edge point (24) on the free margin (22), a first intersecting point (25a) between the free margin (22) and the first margin of attachment (23a), a second intersecting point (25b) between the free margin (22) and the second margin of attachment (23b), and a third intersecting point (26) between the first margin of attachment (23a) and the second margin of attachment (23b). FIG. 5 as shown is a top view of the leaflet component when the leaflets of the leaflet component are partially open and free margins of the three leaflets do not contact each other.

In one embodiment, the valved stent of the present invention comprises a short zone along the free margin (22) around the middle free-edge point (24), wherein the short zone is sized and configured to form a small opening (30), usually about 0.5-3 mm² area, when the free margins (22) of the at least two leaflets (27) coapt to form the one-way flow valve that allows a little transient leakage (bleeding) upon coaptation to minimize the impact of sudden coaptation. The small opening (30) is usually a triangular shape for a trileaflet valved stent.

The leaflet component of the valved stent of the present invention may comprise two, three, four or more leaflets (also known as valvular membranes or cusps). In one embodiment, the leaflet is made of a plastic sheet, such as polytetrafluoroethylene, expanded polytetrafluoroethylene, polyurethane, or other synthetic polymers. Preferably, the leaflet is resistant to thrombosis, anti-thrombotic, or incorporated with anti-thrombosis agent. In another embodiment, the leaflet is made of a tissue sheet selected from the group consisting of bovine pericardium, equine pericardium, ovine pericardium, porcine pericardium, fascia lata, and dura mater.

In one embodiment, the leaflet is made of a decellularized tissue sheet. Quijano and Tu in U.S. patent application Ser. No. 11/704,563 filed on Feb. 9, 2007 and U.S. patent application Ser. No. 11/704,645 filed on Feb. 9, 2007, the entire contents of both are incorporated herein by reference, disclose an exemplary decellularization process. The decellularization process may comprise treating the tissue sheet with a solution containing bile acid or bile salts which effect solubilization of cell membranes of cells present in the tissue sheet. In another embodiment, the leaflet may be treated with a crosslinking agent, wherein the crosslinking agent is selected from a group consisting of genipin, epoxy compounds, dialdehyde starch, glutaraldehyde, formaldehyde, dimethyl suberimidate, carbodiimides, succinimidyls, diisocyanates, acyl azide, and combinations thereof. Quijano and Tu in U.S. patent application Ser. No. 11/897,794, filed Aug. 31, 2007, the entire contents of which are incorporated herein by reference, disclose a self-expanding valve for the venous system.

As shown in FIG. 6, a first cross-sectional area of the outflow section (13b) of the meshed stent component of the valved stent is about the same as a second cross-sectional area of the inflow section (12b). In other words, the meshed stent component is substantially cylindrical all the way from inflow to outflow.

However, as shown in FIG. 7, the outflow section (13a) of the alternate meshed stent component (11a) is bulged that is characterized with a first cross-sectional area of the outflow section of the alternate meshed stent component is larger than a second cross-sectional area of the inflow section (12a). In an exemplary embodiment, the inflow section is cylindrical whereas the outflow section is ovoid or prolate ellipsoid, not spheroid. The bulged outflow section is where a valvular mechanism or leaflet component is located or mounted. In one embodiment, the axial length of the inflow section is about the same or more than twice the length of the outflow section.

In one embodiment, the first margin of attachment of the valved stent of the present invention is mounted between two adjacent, consecutive strut joints on the right-spiral strut. Similarly, the second margin of attachment of the valved stent of the present invention is mounted between two adjacent, consecutive strut joints on the left-spiral strut. In a further embodiment, each leaflet further comprises an extended flat sheet material (28) beyond the first margin of attachment (23a) and second margin of attachment (23b), wherein the first margin of attachment is mounted between two adjacent joints on the right-spiral strut, the second margin of attachment is mounted between two adjacent joints on the left-spiral strut, and wherein the extended flat sheet material is sized and configured for coupling the leaflet onto the strut. The coupling may be by laser fusing, suturing, stapling, ultrasonic fusing or heat (such as radiofrequency) adhesion over around the strut or the covered strut as shown in FIG. 2 and FIG. 4. The extended flat sheet material of the leaflet is solely used for coupling purposes and does not contribute to any usable or effective leaflet area.

In a further embodiment, the first margin of attachment (23a) is mounted between a first and a third joints on the right-spiral strut and the second margin of attachment (23b) is mounted between a first and a third joints on the left-spiral strut. Furthermore, the leaflet of the valved stent of the present invention comprises an extended flat sheet material beyond the first and second margins of attachment, wherein the first margin of attachment is mounted between a first and a third strut joints (as shown in FIG. 3) on the right-spiral strut, the second margin of attachment is mounted between a first and a third strut joints on the left-spiral strut and wherein the extended flat sheet material is sized and configured for coupling the leaflet onto the strut. Likewise, the first margin of attachment (23a) may be mounted between a first and a fourth, fifth or higher-numbered joints on the right-spiral strut as long as a first end of the first margin of attachment meets a second end of the second margin of attachment at the strut joint of the above-mentioned right-spiral strut and the left-spiral strut.

### EXAMPLE NO. 3

Tissue Sheet Trimming as a Leaflet

The valved stent of FIG. 5 shows two margins of attachment (23a, 23b) being sized and configured in a concave shape, the concave-shaped margins of attachment are trimmed or cut from a flat membrane sheet according to illustration of FIG. 8. FIG. 8A shows a trimmed 3-D membrane sheet that is bordered by a free margin (from point 25a to point 25b via point 24), a first margin of attachment (23a) (from point 25a to point 26 following the corresponding spline strut section), and a second margin of attachment (23b) (from point 26 to point 25b following the corresponding spline strut section). At a first specific axial distance upstream from the free margin level (line AA), say H₁, the length of chord (line BB) is measured or calculated between the two points on each of the right-spiral and left-spiral splines at that H₁ level. There is an angle, θ, between either the right-spiral spline (also known as lattice or strut segment) or left-spiral spline (lattice) and an axial reference line. Similarly, at a second specific axial distance upstream from the free margin level (line AA), say H₂, the length of chord (line CC) is measured or calculated between the two points on each of the right-spiral and left-spiral splines at that H₂ level. When one continues, in further steps, to measure or calculate the length of chord (line DD) between the two points on each of the right-spiral and left-spiral splines at that H₃ level and so forth, the concave-shaped margins of attachment (23a and its mirror-image 23b) can be input in an algorithm for computer-controlled membrane cutting or manual calculation as illustrated in Example no. 4.

Some aspects of the invention provide a valved stent (and a method of cutting membrane sheet) with a concave-shaped margin of attachment, wherein the two margins of attachment are sized and configured in a concave shape, the concave-shaped margins of attachment are trimmed with a set of plural pair-data, wherein the pair-data are generated by measuring or calculating the first length of chord between two points on each of the right-spiral and left-spiral struts at a specific first set-distance spaced away upstream from the level of free margin and iteratively until a final set-point coincides with the third intersecting point (26) at the strut joint (16a). By measuring or calculating many pair-data, the leaflet dimension is determined. FIG. 8B shows a leaflet having concave-shaped margins of attachment and their corresponding extended flat material that is cut according to the principles of the present invention on a 2-D flat configuration. The usable, effective sheet area of the leaflet associated in treating chronic venous insufficiency has boundaries within the lines 22, 23a and 23b, wherein the concave-shaped lines or margins of attachment (23a, 23b) are configured according to the algorithm of the present invention (shown in FIG. 8B).

One aspect of the invention provides a valved stent, wherein the concave-shaped first margin of attachment between the first intersecting point and the third intersecting point is trimmed from a tissue sheet following an algorithm derived from the equation of:

T=(S/R)×(180/π),
where T is the length of chord at any specific axial set-point upstream of the free margin, a first end of the chord being the first mounting point on a right-spiral strut and a second end of the chord being the second mounting point on an adjacent left-spiral strut, both mounting points being at the same axial level of that specific set-point.
R is a radius of the valved stent, and
S is the length of chord of the circular arc of the stent cylinder at any specific set-point axially spaced upstream from the free margin.

The leaflet configuration for a stented venous valve is provided herein for the proper fluid vector fields to minimize thrombosis, particularly when the valve is closed and the blood is substantially stagnant. The leaflet configuration as mounted on a tubular meshed stent in a 3-D manner of the present invention controls the leaflet excursion so leaflet would not impact on metal strut and provide even stress over the whole leaflet membrane under various venous hemodynamic flow conditions, from high flow when the valve is open to low flow when the valve is closed and any transient flow in between.

### EXAMPLE NO. 4

Leaflet Dimension Calculation

For a size 20 mm venous valve with trileaflets, the radius R is 10 mm. A set of T and S pair-data can be calculated from T=0 until T reaches the third intersecting point (26) or the strut joint (16a) that is measurable or calculated from the stent strut design. The pair-data are shown in the table below:

SIZE 20 mm TRILEAFLETS
TE = (S/R) × (180/Pi) = S (180/(Pi × 0.394)) = 145.42 × S

T = (145.42/2) × S = 72.71 × S
T1 = 72.71 × 0.825 = 60.00 (M) (.635)
T2 = 72.71 × 0.748 = 54.39 (C) (.064)
T3 = 72.71 × 0.671 = 48.79 (G) (.127)
T4 = 72.71 × 0.594 = 43.19 (Y) (.191)
T5 = 72.71 × 0.517 = 37.59 (M) (.254)
T6 = 72.71 × 0.440 = 32.00 (B) (.318)
T7 = 72.71 × 0.364 = 26.47 (C) (.381)
T8 = 72.71 × 0.287 = 20.87 (G) (.445)
T9 = 72.71 × 0.210 = 15.27 (Y) (.508)
T10 = 72.71 × 0.172 = 12.51 (M) (.540)
T11 = 72.71 × 0.133 = 9.67 (B) (.572)
T12 = 72.71 × 0.095 = 6.91 (C) (.604)
T13 = 72.71 × 0.084 = 6.11 (G) (.613)

The set of many pair-data of T and S are used to provide the X and Y coordinates for cutting a flat membrane sheet of the present invention.

Some aspects of the invention relate to a process for production of the valved stent of the present invention. The process may comprise: (a) providing the cylindrical meshed stent component that has right-spiral struts, left-spiral struts and a strut joint at a crossing of any of the right-spiral struts and any of the left-spiral struts; (b) providing a tissue sheet that is trimmed according to the algorithm as shown in Example No. 3; (c) coupling the first margin of attachment between two adjacent strut joints on the right-spiral strut; and (d) coupling the second margin of attachment between two adjacent strut joints on the left-spiral strut.

From the foregoing description, it should now be appreciated that a novel and unobvious valved stent configured for use in a venous low pressure environment has been disclosed for treating chronic venous insufficiency and medical applications. While the invention has been described with reference to a specific embodiment, the description is illustrative of the invention and is not to be construed as limiting the invention. Various modifications and applications may occur to those who are skilled in the art, without departing from the true spirit and scope of the invention.

What is claimed is:

1. A valved stent comprising: a cylindrical meshed stent component with lattices, said stent component having an inflow section and an outflow section with respect to flow direction, each of the inflow and outflow sections having right-spiral struts, left-spiral struts and a strut joint at a crossing of any of said right-spiral struts and any of said left-spiral struts; and a leaflet component with at least two identical leaflets, each leaflet having a free margin and two margins of attachment, wherein a first margin of attachment is mounted on a right-spiral strut and a second margin of attachment is mounted on an adjacent left-spiral strut, wherein a first end of the first margin of attachment meets a second end of the second margin of attachment at the strut joint of the above-mentioned right-spiral strut and the left-spiral strut, the free margins of said at least two leaflets coapting to form a one-way flow valve.

2. The valved stent of claim 1, wherein the leaflet comprises a middle free-edge point on said free margin, a first intersecting point between the free margin and the first margin of attachment, a second intersecting point between the free margin and the second margin of attachment, and a third intersecting point between the first and the second margins of attachment.

3. The valved stent of claim 2, wherein a short zone along the free margin around the middle free-edge point is configured to form a small opening when the free margins of said at least two leaflets coapt to form the one-way flow valve that allows a little transient leakage, wherein the small opening is about 0.5 to 3 mm² area.

4. The valved stent of claim 2, wherein the two margins of attachment are sized and configured in a concave shape, said concave-shaped margins of attachment are trimmed with a set of plural pair-data, wherein said pair-data are generated by measuring or calculating the first length of chord between two points on each of the right-spiral and left-spiral struts at a specific first set-distance spaced away upstream from level of the free margin and iteratively until a final set-point coincides with the third intersecting point.

5. The valved stent of claim 4, wherein the concave-shaped first margin of attachment between the first intersecting point and the third intersecting point is trimmed from a tissue sheet having X and Y coordinates by following an algorithm derived from the equation of:
T=(S/R)×(180/π),
where T is the length of chord at any specific axial set-point upstream of the free margin, a first end of the chord being the first mounting point on a right-spiral strut and a second end of the chord being the second mounting point on an adjacent left-spiral strut, both mounting points being at the same axial level of that specific set-point.
R is a radius of the valved stent, and
S is the length of chord of the circular arc of the stent cylinder at any specific set-point axially spaced upstream from the free margin.

6. The valved stent of claim 1, wherein said stent is configured for treating chronic venous insufficiency.

7. The valved stent of claim 1, wherein said leaflet component comprises three leaflets.

8. The valved stent of claim 1, wherein said leaflet is made of a tissue sheet selected from the group consisting of bovine pericardium, equine pericardium, ovine pericardium, porcine pericardium, fascia lata, and dura mater.

9. The valved stent of claim 1, wherein said leaflet is made of a decellularized tissue sheet.

10. The valved stent of claim 1, wherein the tissue sheet is treated with a solution containing bile acid or bile salts which effect solubilization of cell membranes of cells present in said tissue sheet.

11. The valved stent of claim 1, wherein said leaflet is treated with a crosslinking agent.

12. The valved stent of claim 1, wherein said leaflet is treated with a crosslinking agent, wherein the crosslinking agent is selected from a group consisting of genipin, epoxy compounds, dialdehyde starch, glutaraldehyde, formaldehyde, dimethyl suberimidate, carbodiimides, succinimidyls, diisocyanates, acyl azide, and combinations thereof.

13. The valved stent of claim 1, wherein a first cross-sectional area of the outflow section of said meshed stent component is about the same as a second cross-sectional area of the inflow section, wherein an axial length of the inflow section is about twice or more of a length of the outflow section.

14. The valved stent of claim 1, wherein the outflow section of said meshed stent component is bulged that is characterized with a first cross-sectional area of the outflow section of said meshed stent component is larger than a second cross-sectional area of the inflow section.

15. The valved stent of claim 1, wherein the first margin of attachment is mounted between two adjacent strut joints on said right-spiral strut.

16. The valved stent of claim 1, wherein said leaflet further comprises an extended flat sheet material beyond the first and second margins of attachment, wherein the first margin of attachment is mounted between two adjacent joints on said right-spiral strut and wherein the extended flat sheet material is sized and configured for coupling said leaflet onto said strut, wherein said extended flat sheet material does not contribute to any effective leaflet area.

17. The valved stent of claim 1, wherein the first margin of attachment is mounted between a first and a third joints on said right-spiral strut.

18. The valved stent of claim 1, wherein said leaflet further comprises an extended flat sheet material beyond the first and second margins of attachment, wherein the first margin of attachment is mounted between a first and a third strut joints on said right-spiral strut and wherein the extended flat sheet material is sized and configured for coupling said leaflet onto said strut.

19. The valved stent of claim 1, wherein said leaflet is a plastic sheet.

20. A process for production of the valved stent of claim 1, comprising:
providing the cylindrical meshed stent component that has right-spiral struts, left-spiral struts and a strut joint at a crossing of any of said right-spiral struts and any of said left-spiral struts;
providing a tissue sheet that is trimmed according to the algorithm of claim 5;
coupling the first margin of attachment between two adjacent strut joints on said right-spiral strut; and
coupling the second margin of attachment between two adjacent strut joints on said left-spiral strut.
